(19) United States
(12) Patent Application Publication
(10) Pub. No.: US 2008/0069776 A1
(43) Pub. Date: Mar. 20, 2008

(54) HISTOFLUORESCENT STAIN FOR ENDOSCOPY

(75) Inventors: Akira YAMAMOTO, Tokyo (JP); Yusuke IIMORI, Tokyo (JP); Mizue SAZE, Tokyo (JP); Mariko ISHIGURO, Tokyo (JP); Yae KUROSAWA, Tokyo (JP); Hiroyuki SASAKI, Kanagawa (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/855,254

(22) Filed: Sep. 14, 2007

(30) Foreign Application Priority Data
Sep. 14, 2006 (JP) 2006-248849

(51) Int. Cl. A61B 1/06; A61K 49/00

(52) U.S. Cl. 424/9.6; 600/160; 600/176

(57) ABSTRACT

A histofluorescent stain for endoscopy having good fluorescent stainability for the inner part of tissues is provided.

A histofluorescent stain composition for endoscopy, containing a compound represented by the following Formula (1)

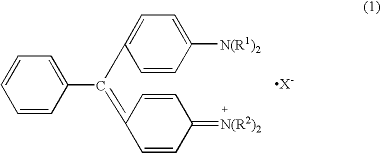

wherein R¹ and R², which may be identical or different, each represent an alkyl group having 1 to 5 carbon atoms; and X⁻ represents an anion residue.

### BACKGROUND OF THE INVENTION

1. Field of the Invention

The present invention relates to a histofluorescent stain composition used in diagnoses by endoscopy.

2. Description of the Related Art

Diagnostic techniques employing endoscopy are widely used in the diagnoses of diseases such as, in particular, cancers, gastrointestinal ulcers and ulcerative colitis, on the basis of the gastrointestinal endoscopic examination for the upper gastrointestinal tract and the lower gastrointestinal tract. The detection of anomalies (lesions) in the tissues by such endoscopic examination generally involves observation with a visible light endoscope at a magnification of about 10 to 500 times without using a staining agent. Meanwhile, there is a method referred to as chromoendoscopy, in which endoscopic observation is made in the state where a solution containing a dye has been spread over tissue surfaces. Since it is possible to clearly observe the morphology of the surface of the gastrointestinal lumen by this chromoendoscopy, even microscopic lesions can be easily discovered by the change of color tone. Examples of the endoscope that can be used for chromoendoscopy include a visible light endoscope and a fluorescent endoscope.

Examples of the dye mainly used in chromoendoscopy include indigo carmine (Masahiro Tada, Akitada Iso, et al., (Clinical Gastroenterology, vol. 7, No. 2, 1992)), which is used for observation by the contrast method, for the staining of the gastrointestinal lumen under visible light; acriflavin and fluorescein (Gastroenterology 2004, vol. 127, No. 3, p. 706-713) for fluorescent staining; and the like.

In the diagnosis of cancer or the like, it is important to conduct observation of the surface of biological tissues as well as the inner part of biological tissues. With regard to the method of observing the inner part of biological tissues, it is general to use a method of slicing a micro part of a tissue collected by biopsy or the like in the laboratory, and staining and observing the micro part. For the method of observing the inner part of biological tissues in situ, for example, MRI, PET, CT, soft X-ray method and the like are applied for the observation of the whole body. With regard to the gastrointestinal endoscopy, endoscopes involving autofluorescent reaction of biological tissues have been commercialized. When a light having a specific wavelength is irradiated to a biological tissue, there occurs autofluorescence of endogenous fluorescent substances in the tissue, and thus, visual observation of normal areas and lesion areas can be made possible by means of the intensity differences and the spectra.

In order to make a diagnosis of, for example, gastrointestinal cancer using a conventional endoscope, it has been required to determine the lesion area in an empirical manner by observation, excise a tissue specimen therefrom, and make a diagnosis by means of tissue staining in a separate laboratory. However, it is now possible to observe the inner part of a tissue without excising the tissue, by confocal endoscope, which has been developed recently.

In general, a confocal imaging system is based on a technique of obtaining clear images of the inner part of a tissue without mechanical cutting, by placing a pinhole in front of the detector and detecting the light reflected only from the focal plane inside the tissue. Typically, the confocal image system involves scanning a laser light to a tissue stained with a fluorescent substance, and observing a fluorescent image thereof. The confocal imaging system generally needs fluorescent staining agents.

Since the confocal endoscope employing the confocal imaging system has both a normal observation optical system and a confocal observation optical system, it is useful from the viewpoints that screening of the lesion area is possible, and that the observation of cells through optical tissue slicing without cutting out the cells is less invasive as well as possible in situ.

Confocal endoscopes that are currently being marketed employ blue laser light having a wavelength of 488 nm as a light source for dye excitation. It is an important property required for a fluorescent dye to be used in the confocal endoscopes for medical purposes that the fluorescent dye has no toxicity or mutagenecity against the body. Therefore, for the present, the fluorescent dye that can be used for confocal endoscopes for medical purposes is limited to fluorescein for intravenous injection for fundus angiographies, and acriflavin that is used as an antibiotic substance (Gastrointestinal Endoscopy Clin of N Am, 2005, vol. 12, p. 715-731).

With regard to the light source used for the confocal endoscopes, diversification of the wavelength is anticipated in the future. For example, since a light in the region from red to infrared has higher permeability to biological tissues than blue light, it is believed to give confocal images at positions deeper than the surface of the biological tissues. Indocyanine Green (Icg) is available as an infrared fluorescent compound for diagnosis which has been approved for administration to human body, and it is mainly used for the examination of hepatic function and fundus angiography. If Indocyanine Green is used as a staining agent for confocal endoscopy, it is not possible to achieve clear contrast compared to, for example, the fluorescein described in Gastroenterology 2004, vol. 127, No. 3, p. 706-713. Furthermore, if Indocyanine Green is to be used as a fluorescent stain for endoscopy, it has a problem of being highly toxic compared to the fluorescein.

### SUMMARY OF THE INVENTION

Accordingly, it is an object of the present invention to provide a histofluorescent stain composition for endoscopy, which composition 1) has low biotoxicity, 2) confers contrast to emphasize the irregularities on tissues even under a white light source, 3) emits fluorescence, and 4) has good fluorescent stainability for the inner part of tissues and is useful for diagnosis of lesions in the inner part of tissues.

The inventors of the present invention have investigated in various aspects while focusing on the high safety of a dye, and as a result, found that a compound of the following Formula (1), which is represented by Brilliant Green, satisfies the requirements described above. In addition, they also discovered that such compound is useful as a fluorescent stain for selective staining of the inner part of a tissue, particularly since the compound does not emit fluorescence in an aqueous solution, but emits fluorescence only when applied to a tissue, in particular to albumin, thereby yielding clear fluorescent images of the inner part of the tissue. Thus, the inventors have now completed the present invention based on this finding.

Thus, the present invention is to provide a histofluorescent stain composition for endoscopy, comprising a compound represented by the following Formula (1):

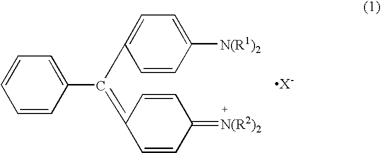

wherein R¹ and R², which may be identical or different, each represent an alkyl group having 1 to 5 carbon atoms; and X⁻ represents an anion residue.

It is another object of the present invention to provide use of the compound represented by Formula (1), for the manufacture of a histofluorescent stain for endoscopy.

It is still another object of the present invention to provide a method of diagnosing with an endoscope by administering a composition comprising the compound represented by Formula (1), and observing the tissue stained by the composition, with an endoscope.

(1) For the observation of the gastrointestinal tract under a conventional endoscope using a white light source, the histofluorescent stain of the present invention, which is a blue dye, results in strong contrast compared to red dyes such as fluorescein.

(2) For the observation with an endoscope which combines a white light endoscope and a confocal endoscope, the stain of the present invention confers blue contrast with respect to white light, while exhibiting tissue-specific fluorescence with respect to red exciting light. Thus, upon using the endoscope described above, the discovery of lesion areas and the obtainment of confocal images thereof can be provided by a single dye.

(3) The compound of Formula (1) does not exhibit fluorescence in an aqueous solution, but exhibits fluorescence only when applied to a tissue, in particular to albumin. Accordingly, the compound does not stain the interstitial space between cells, and can specifically stain the inner part of the tissue.

(4) Accordingly, when the histofluorescent stain of the present invention is used, visualization of the inner part of a lesion tissue can be achieved simultaneously, without extracting the tissue, under observation by visible light, fluorescence and confocal endoscopies, and the stained images are sharp and clear. Thus, the stain is useful in the diagnosis of gastrointestinal diseases and the like.

### BRIEF DESCRIPTION OF THE DRAWINGS

FIG. 1 is a diagram illustrating the excitation (Abs.) and fluorescence (Emission) spectrum of Brilliant Green;

FIG. 2 is a diagram illustrating the excitation (Abs.) and fluorescence (Emission) spectrum of Rhodamine B;

FIG. 3 is a diagram illustrating the fluorescence spectrum of a liquid mixture of Brilliant Green and albumin;

FIG. 4 is a diagram illustrating the fluorescent spectrum of albumin;

FIG. 5 is a set of photographs showing the results of staining of an area 35 μm deep from the luminal surface layer of the colon, which was fluorescent stained with Brilliant Green;

FIG. 6 is a photograph showing the results of staining of an area 10 μm deep from the luminal surface layer of the colon, which was fluorescent stained with Brilliant Green; and

FIG. 7 is a photograph showing the results of staining of an area 15 μm deep from the luminal surface layer of the colon, which was fluorescent stained with Brilliant Green.

### DESCRIPTION OF THE PREFERRED EMBODIMENTS

The present disclosure relates to subject matter contained in Japanese Patent Application No. 2006-248849 (filed on Sep. 14, 2006) which is expressly incorporated herein by reference in its entirely.

Examples of the endoscopy according to the present invention include medical endoscopies such as gastrointestinal endoscopy, respiratory endoscopy, vascular endoscopy and peritoneoscopy. Among these, gastrointestinal endoscopy is particularly preferred. According to the present invention, the visible light endoscopes include all of the endoscopes observing under visible light, and also include conventional endoscopes, magnifying endoscopes, and chromoendoscopes observing visible light. Meanwhile, the fluorescent endocopes include endoscopes measuring the fluorescence generated by irradiation of an excitation light, and also include magnifying fluorescent endoscopes. Furthermore, the confocal endoscope refers to an endoscope equipped with a confocal imaging system. In addition, a confocal endoscope conventionally has both a normal observation optical system and a confocal observation optical system.

The histofluorescent stain composition of the present invention contains a compound represented by the Formula (1) described above. In the Formula (1), R¹ and R², which may be identical or different, each represent an alkyl group having 1 to 5 carbon atoms. Examples of the alkyl group include a methyl group, an ethyl group, an n-propyl group, an isopropyl group, an n-butyl group, and the like, and among these, a methyl group and an ethyl group are preferred. Furthermore, it is particularly preferred when both R¹ and R² represent an ethyl group.

X⁻ represents an anion residue, and may be exemplified by HOSO₃⁻ (sulfate ion), a halogen ion, or the like. Among these, HOSO₃⁻ is particularly preferred.

As a specific preferred example of the compound of Formula (1), Brilliant Green (R¹═R²═C₂H₅, X⁻═HOSO₃⁻) may be included.

Brilliant Green is widely used as a colorant for cosmetic products. It is a legal colorant that can be used in cosmetic products. The safety of this component has been established. However, it is not fully known that this compound emits fluorescence only when bound to a protein such as albumin, and it is not known at all that the compound exhibits clear fluorescent images of the inner part of a tissue, only when applied to a tissue.

Commercially available products of the Brilliant Green include, for example, Brilliant Green (B4014-25G) from Sigma-Aldrich Company. It is also available under different names such as Pigment Green 1 and Basic Green, and is indicated by C.I. (Color Index Number) 42040.

The content of the compound of Formula (1) in the tissue stain of the present invention is preferably from 0.01 to 70% by weight, more preferably from 0.01 to 50% by weight, and particularly preferably from 0.01 to 20% by weight, from the viewpoints of stainability and the clearness of stained images.

The tissue stain of the present invention can be used in any form, such as liquid, granule, or tablet. In the case of spreading into the gastrointestinal tract or administering submucosally, liquid is preferred, while in the case of orally administering, liquid, granules, tablets and the like are preferred.

The tissue stain of the present invention may have various components blended in according to the form (formulation). For example, a consistency agent, a thickening agent, a surfactant, a sweetening agent, a preservative, a flavoring agent, a pH adjusting agent, water and the like may be blended in.

The pH adjusting agent is an agent adjusting the pH to 5 to 9, and examples thereof include hydrochloric acid, phosphoric acid, citric acid, malic acid, acetic acid and salts thereof, sodium hydroxide, potassium hydroxide, sodium hydrogen carbonate, tetrasodium pyrrolate, and the like. Furthermore, ethanol, water and the like may be added as a solvent. In the case of tablets, components that are known to be used for tablets, such as a binding agent and a disintegrating agent, can be used.

Since the tissue stain of the present invention can stain tissues to be blue in color, the stain is useful as a tissue stain for conventional white light endoscopic observation. The endoscope used herein is a conventional endoscope or a magnifying endoscope, and is useful for endoscopic observation at a magnifying power of 10 to 500 times.

The stainability for tissues varies, from the state of normal tissues to a pre-cancerous state or a state in the presence of tumor. In the case of staining normal tissues, the cellular membrane and cytoplasm of the epithelial cells are stained in the small intestine or the large intestine. On the other hand, in the observation of tumors, features of individual cells include an increase in the nuclear volume, an increase in the amount of nuclear chromatin and hyperchromatism, an increase in the number of nucleoli, abnormality in the number or form of chromosomes, basophilic staining of cells and its association with the proliferation properties of the cells, and the like. Furthermore, in tumor cells, a large number of nuclear chromatins assemble close to the nuclear membrane, thus endoplasmic reticulum being simplified. Moreover, mitochondria become irregular and non-uniform in size, and there is an increased amount of filamentous structures generated in the cells.

The tissue stain composition of the present invention has been found, first of all, to provide good staining of the epithelial cells and the cytoplasm while practically not staining the interstitial space, by observation. Therefore, the morphology of cells in the tissue or the characteristics of cellular arrangement can be detected. The stain composition will be highly useful with an endoscope utilizing the wavelength range which is adequate for the stain composition, since abnormality in tissues and cells is suited for the observation by a confocal imaging system. Furthermore, since the cellular nucleus is not stained, determination can be made more easily in the case of the pre-cancerous state described above, and the like.

Also, the tissue stain composition of the present invention has a property of emitting fluorescence only for bound tissues, and thus necessary information can be obtained even without washing by running a solution. From this point of view, it can also be said that the significance of the information obtained by the stain of the present invention is high.

As indicated by the following Examples, the compound of Formula (1) is characterized in that, as compared to other dyes, the compound does not emit fluorescence in a solution state, but emits fluorescence when spread in the lumen of the body, and that the compound provides clear fluorescence stained images of the inner part of tissues. Therefore, the stain of the present invention is very useful as a histofluorescent stain for endoscopy.

In addition, if the endoscope employing a confocal optical system has both a normal observation optical system and a confocal observation optical system, it is possible to diagnose the surface as well as the inner part of tissues without excising the tissue in the lesion area, by visually observing the lesion area by observation under normal light, and then upon reaching the lesion of question, observing fluorescent stained cross-sectional images of the inner part of the tissue (for example, up to 250 μm) with a confocal endoscope. That is, the morphology of cells or nuclei of a biological tissue can be observed in a live state. As a result, diagnosis of gastrointestinal diseases such as pre-cancerous state, cancer, ulcer, and ulcerative colitis is made possible safely, rapidly and low-invasively, and the degree of precision is remarkably improved.

With regard to such endoscopic observation, the tissue stain of the present invention may be directly spread in the gastrointestinal lumen or administered submucosally, or may be administered orally or intravenously.

### EXAMPLES

Next, the present invention will be described in more detail with reference to Examples, but the present invention is not intended to be limited by these Examples.

### Example 1

Brilliant Green (manufactured by Sigma-Aldrich Company, B4014-25G) was adjusted with distilled water to a concentration of 0.001 mg/mL, and the light absorbance was continuously measured over a wavelength range from 200 to 750 nm using a spectrophotometer (manufactured by Shimadzu Corporation, BioSpec-1600) to determine the wavelength at which the absorption was the maximum. The maximum absorption wavelength of 625 nm thus obtained was irradiated as an excitation wavelength, and the wavelength of the scattered light detected in the perpendicular direction to the light axis of the excitation light was measured using a fluorescence spectrophotometer (manufactured by Shimadzu Corporation, RF-1500), thus to obtain a maximum fluorescence wavelength of 625 nm. The maximum excitation wavelength measured using this fluorescence spectrophotometer was 627 nm. The FIG. 1 shows the maximum excitation wavelength and the maximum fluorescence wavelength measured with the fluorescence photometer.

As a result, no fluorescence was detected from the stain which was prepared as a solution. The same test was performed using Brilliant Blue FCF, which is widely used as an edible blue dye No. 1, and as a result, Brilliant Blue FCF was found to have a maximum absorption wavelength of 629 nm, a maximum excitation wavelength of 619 nm, and a maximum fluorescence wavelength of 650 nm, showing a clear difference in the fluorescence intensity in the same wavelength region. Brilliant Green was found to emit no fluorescence under a light source in the red wavelength region.

When measured with a fluorescence photometer, Brilliant Green does not show any difference in the maximum absorption wavelength and the maximum fluorescence wavelength. This implies that the compound is not undergoing the process of excitation by an irradiated light and emission of fluorescence.

However, when a biological tissue from the large intestine or the like is stained with Brilliant Green and excited under a certain laser light, observation by means of intense fluorescence can be made.

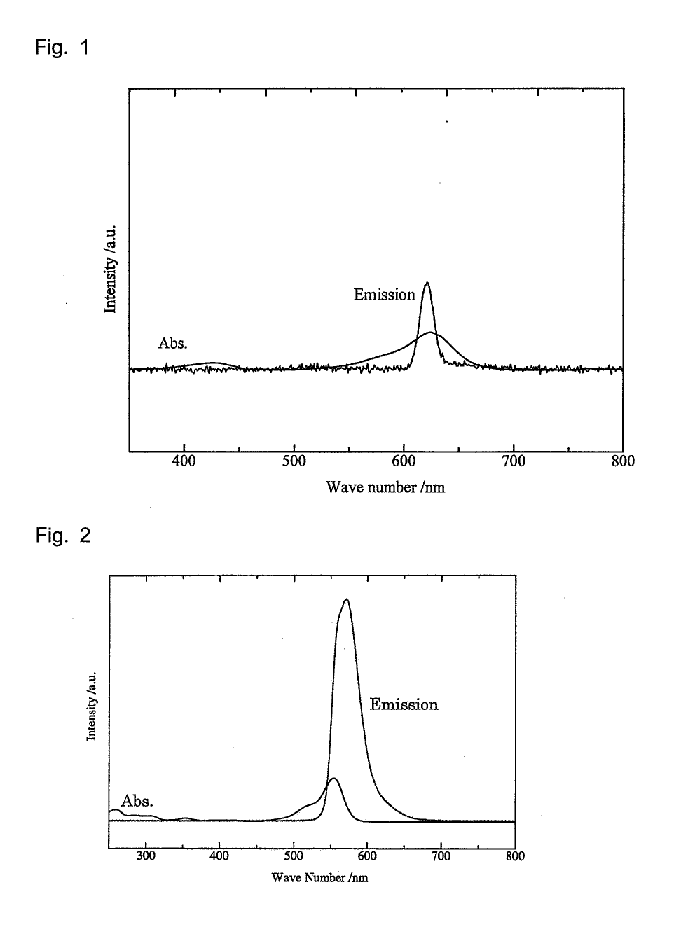

As a comparison to Brilliant Green, the wavelength pattern of Rhodamine B, which stains the inner part of cells in the lumen of the large intestine or the like in the same manner as Brilliant Green does, was measured. It could be confirmed that Rhodamine B exhibits intense fluorescence even in a solution state, as shown in FIG. 2.

### Example 2

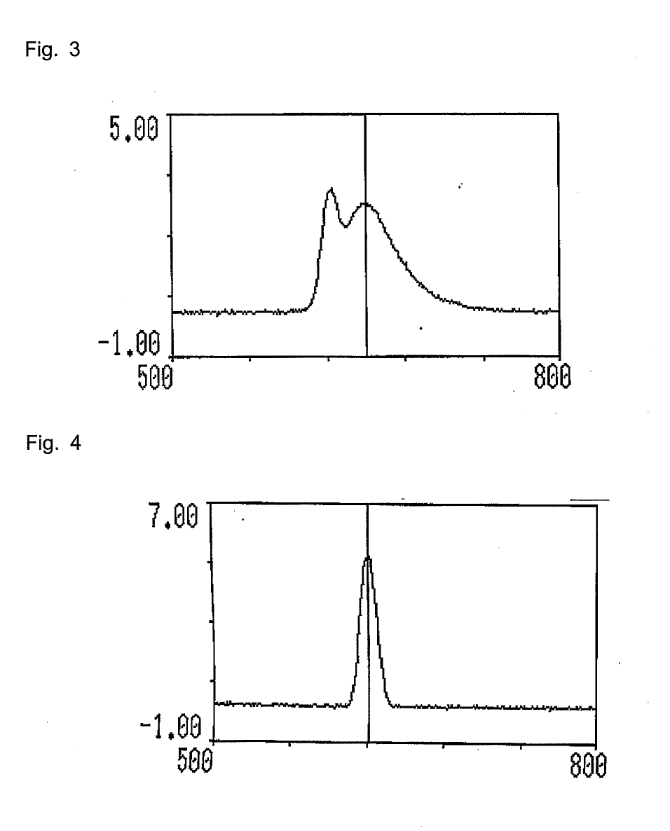

Brilliant Green (manufactured by Sigma-Aldrich Company, B4014-25G) was prepared as a solution at 0.1 mg/mL using distilled water. Albumin (from Bovine Serum, Globulin Free; Wako Pure Chemical Industries, Ltd., 013-15104) was prepared as a solution at 2.0 mg/mL using physiological saline. Using a 0.1 M phosphate buffer solution (pH 5) and the 2.0 mg/mL albumin solution prepared above, a liquid mixture of 0.01 mg/mL Brilliant Green and albumin was prepared. The maximum absorption wavelength of Brilliant Green of 625 nm was irradiated as the excitation wavelength, and the wavelength of the scattered light detected in the perpendicular direction to the light axis of the excitation light, was measured using a spectrophotometer (manufactured by Shimadzu Corporation, RF-1500). The maximum fluorescence wavelength was 651 nm. FIG. 3 shows the maximum fluorescence wavelength measured by a fluorescence photometer.

As a comparison to the liquid mixture of Brilliant Green and albumin, the maximum fluorescence wavelength of an albumin solution was measured with respect to an excitation wavelength of 625 nm, which was shown in FIG. 4. Since the spectrum does not show any difference in the excitation wavelength and the fluorescence wavelength, it was found that albumin itself did not emit fluorescence at an excitation wavelength of 625 nm.

From these, it was determined that although Brilliant Green does not emit fluorescence in the state of an aqueous solution, Brilliant Green emits fluorescence, when bound to albumin, under a light source in the red wavelength region.

### Example 3

Brilliant Green (manufactured by Sigma-Aldrich Company, B4014-25G) was prepared as a solution using distilled water, and the concentration was adjusted to 1.0 mg/mL.

A mouse (ddY, 13 weeks old, male) was subjected to laparotomy under anesthesia, and the large intestine was extracted. Immediately after extraction, the prepared solution of Brilliant Green was applied onto the large intestine, and fluorescence observation of the specimen by a confocal imaging system using a confocal microscope (manufactured by Leica Corp., TCPSP2) was performed. Here, the stainability, penetrability, and fluorescence properties of Brilliant Green were examined.

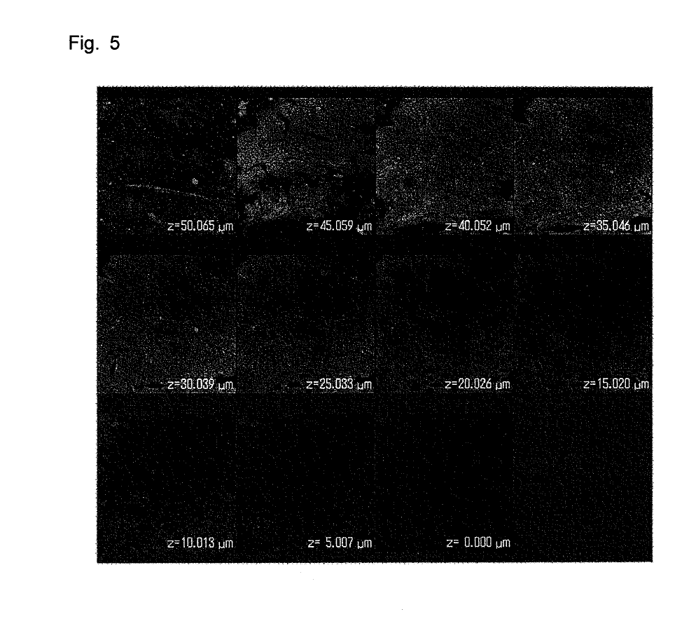

Observation was performed for the cross-sections at every 5 μm from the luminal surface layer of the flesh specimen of extracted large intestine, to the depth direction (FIG. 5).

As can be seen from FIG. 5, observation could be sufficiently made to a depth of 35 μm. Thus, it is also possible to make observation in the depth direction, by adjusting gain values and the like.

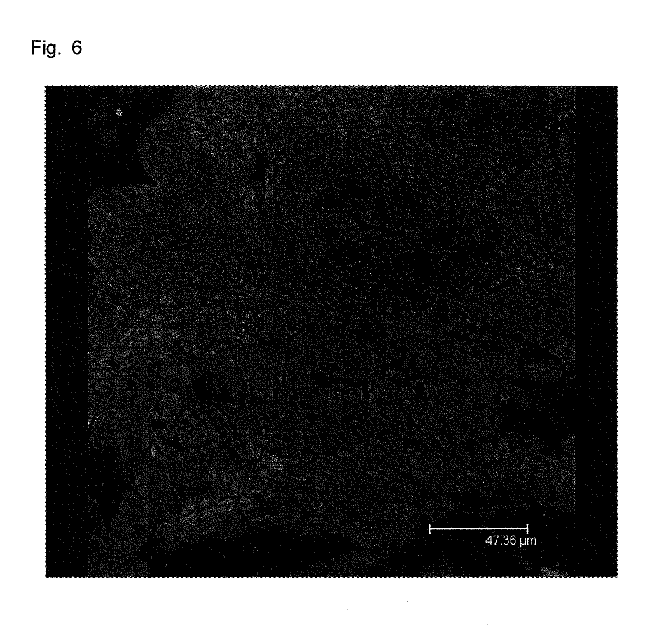

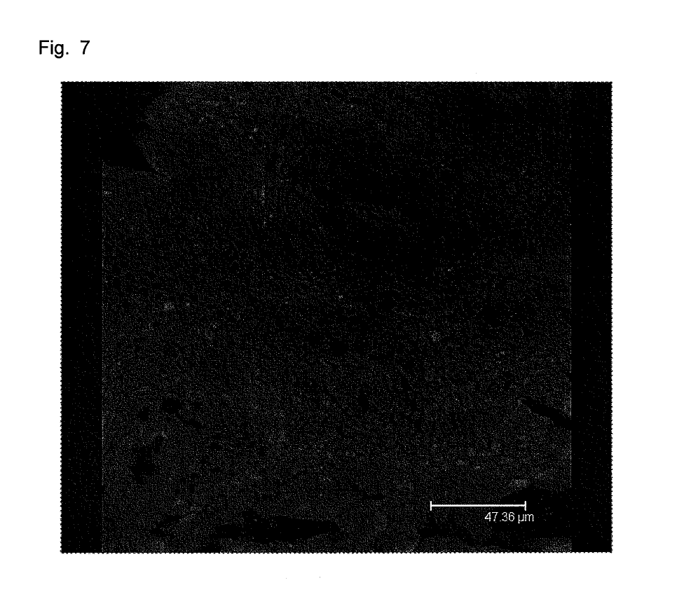

FIG. 6 is a cross-sectional image at a site 10 μm deep from the surface layer, and FIG. 7 is a cross-sectional image at a site 15 μm deep from the surface layer. The cytoplasm is well stained, while the interstitial space is contrastively dark. Thus, very good observation of the morphology or condition of cells can be made.

Unless otherwise stated, a reference to a compound or component includes the compound or component by itself, as well as in combination with other compounds or components, such as mixtures of compounds.

As used herein, the singular forms “a,” “an,” and “the” include the plural reference unless the context clearly dictates otherwise.

Except where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention, At the very least, and not to be considered as an attempt to limit the application of the doctrine of equivalents to the scope of the claims, each numerical parameter should be construed in light of the number of significant digits and ordinary rounding conventions.

Additionallly, the recitation of numerical ranges within this specification is considered to be a disclosure of all numerical values and ranges within that range. For example, if a range is from about 1 to about 50, it is deemed to include, for example, 1, 7, 34, 46.1, 23.7, or any other value or range within the range.

1. A method of diagnosing with an endoscope, comprising administering a composition containing a compound represented by the following Formula (1): 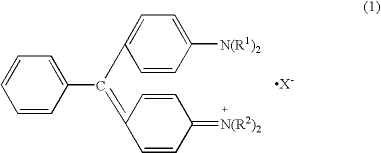
wherein R¹ and R², which may be identical or different, each represents an alkyl group having 1 to 5 carbon atoms; and X⁻ represents an anion residue,
and observing the tissue stained by the composition using an endoscope.

2. The method of diagnosing according to claim 1, wherein
R¹ and R² are each an ethyl group.

3. The method of diagnosing according to claim 1, wherein
X⁻ is HOSO₃⁻.

4. The method of diagnosing according to claim 1, wherein
the endoscope is a confocal endoscope.

5. The method of diagnosing according to claim 1, wherein
the administration of the composition is achieved by oral administration, direct administration into the gastrointestinal tract, or submucosal administration.

6. The method of diagnosing according to claim 1, wherein
the surface of the gastrointestinal lumen and/or the inner part of the cells of the gastrointestinal lumen is stained.

7. A histofluorescent stain composition for endoscopy, comprising a compound represented by the following Formula (1): 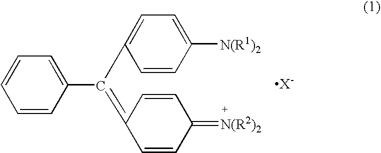
wherein R¹ and R², which may be identical or different, each represent an alkyl group having 1 to 5 carbon atoms; and X⁻ represents an anion residue.

8. The histofluorescent stain composition according to claim 7, wherein
R¹ and R² are each an ethyl group.

9. The histofluorescent stain composition according to claim 7, wherein
X⁻ is HOSO₃⁻.

10. The histofluorescent stain composition according to claim 7, wherein
the endoscope is a confocal endoscope.
